(19) United States
(12) Patent Application Publication
(10) Pub. No.: US 2024/0238343 A1
(43) Pub. Date: Jul. 18, 2024

(54) PHARMACEUTICAL COMPOSITION FOR PREVENTING OR TREATING BRAIN AND NERVOUS SYSTEM DISEASES

(71) Applicant: THE CATHOLIC UNIVERSITY OF KOREA INDUSTRY-ACADEMIC COOPERATION FOUNDATION, Seoul (KR)

(72) Inventors: Sung Won KIM, Seoul (KR); Do Hyun KIM, Seoul (KR); Jung Yeon LIM, Seoul (KR); Sun Hwa PARK, Seoul (KR); Jung Ho CHUN, Seoul (KR); Sin-Soo JEUN, Seoul (KR); Soon A PARK, Seoul (KR)

(21) Appl. No.: 18/004,562

(22) PCT Filed: Jul. 1, 2021
(86) PCT No.: PCT/KR2021/008371

§ 371 Date: Jan. 6, 2023

(30) Foreign Application Priority Data
Jul. 7, 2020 (KR) 10-2020-0083640

(51) Int. Cl. A61K 35/28; A61K 9/00; A61K 9/12; A61P 25/28

(52) U.S. Cl. CPC A61K 35/28; A61K 9/0019; A61K 9/0043; A61K 9/0085; A61K 9/12; A61P 25/28

(57) ABSTRACT

The present invention relates to a pharmaceutical composition comprising nasal inferior turbinate-derived mesenchymal stem cells as an active ingredient, the composition being administered by intranasal or intramaxillary administration by means of olfactory mucosa subepithelial injection, and the like, in order to bypass the blood-brain barrier through a peri-olfactory pathway. The present invention relates to a method in which stem cells, and the like are injected intranasally, thereby enabling various therapeutic materials including the stem cells to be efficiently delivered to the brain and nervous system through the olfactory organ and a trigeminal nervous system pathway, and thus, through the method, it was confirmed that side effects occurring as a result of an entire body being exposed to a drug may be reduced, and further, a sufficient amount of inferior turbinate-derived mesenchymal stem cells may be obtained safely at a desired time at low cost with high efficiency, and it was confirmed that since the composition shares the same genetic origin with the composition-administered subject, the composition exhibits an excellent effect which is the same as or greater than the effects of bone marrow-derived mesenchymal stem cells or adipose-derived mesenchymal stem cells with fewer side effects, and thus, by intranasally administering various therapeutic materials including the nasal inferior turbinate-derived mesenchymal stem cells, the composition is expected to be capable of minimizing side effects and utilized for preventing or developing a treatment for brain and nervous system diseases.

### CROSS-REFERENCE TO RELATED APPLICATIONS

This application is a national phase application filed under 35 U.S.C. § 371 claiming benefit to International Patent Application No. PCT/KR2021/008371, filed Jul. 1, 2021, which claims the benefit of priority from Korean Patent Application No. 10-2020-0083640, filed Jul. 7, 2020, the contents of each of which are hereby incorporated by reference in their entirety.

### TECHNICAL FIELD

The present invention relates to a pharmaceutical composition for preventing or treating brain and nervous system diseases, and more particularly, to a pharmaceutical composition for preventing or treating brain and nervous system diseases, including nasal inferior turbinate-derived mesenchymal stem cells as an active ingredient, and a pharmaceutical composition for preventing and treating brain and nervous system diseases by bypassing the blood-brain barrier through administration to areas including the nasal septum, the olfactory mucosal epithelium, the superior turbinate adjacent to the olfactory mucosal epithelium and the middle turbinate through the nostril or maxillary sinus to maximize the delivery yield of the pharmaceutical composition.

### BACKGROUND ART

As people live longer and become older, brain and nervous system diseases such as stroke, dementia, and Parkinson's disease are increasing. The brain and nervous system diseases are characterized in that the death or degeneration of specific brain cells progresses temporarily or over a long period of time, but once brain cells are dead, they are not regenerated, thereby leading to the ultimate fatal loss of brain function. In particular, brain dysfunction accompanied by progressive deterioration of cognitive, sensory, motor, and systemic functions ultimately brings changes in personality and behavior, leading to a situation in which patients are unable to take care of themselves.

For the successful treatment of neurological disorders as described above, it is very important to efficiently deliver drugs to the brain and nervous system. However, since the brain and nervous system tissue is surrounded by the hard skull and the blood-brain barrier (BBB), the delivery efficiency of therapeutic drugs is very low. Further, in the case of intravenous or arterial administration, which is an existing general drug administration method, there is a problem in that a drug needs to be administered in a larger amount than the drug required for an affected area and there are side effects due to this.

Meanwhile, recently, while the study and technology in the field of treatment of brain and nervous system diseases using stem cells has remarkably developed, the number of cases of actual clinical application has increased, so that stem cell therapy has become an important field for brain and nervous system diseases. Although human embryonic stem cells and dedifferentiated stem cells with high proliferative potential and pluripotency have attracted much attention in therapeutic research of brain and nervous system diseases using stem cells, currently, there are safety, ethical issues, and the like involved in using these stem cells. Under these circumstances, many researchers are interested in adult stem cells as an alternative to embryonic stem cells, and among them, mesenchymal stem cells, which have high self-renewal ability and the ability to differentiate into various tissue cells, are used for various purposes such as research and treatment.

In particular, although mesenchymal stem cells may be isolated from various tissues such as bone marrow, umbilical cord blood, and adipose tissue, and are very useful cells for clinical application because mesenchymal stem cells are safe and have no ethical issues, there are few clinical tests that have confirmed the effective therapeutic effect of stem cell therapeutic agents, and the amount of mesenchymal stem cell supply sources is limited, and there is a problem in that the characteristics of cells may be lost depending on the state of the supply source and the degree of culture. Furthermore, an operation for obtaining mesenchymal stem cells may be accompanied by severe pain, or may need general or spinal anesthesia, and has brought problems in that the amount of obtained mesenchymal stem cells is very small, much time and costs are required while a clinically sufficient amount thereof is cultured, and the risk of contamination and cell loss is high. Therefore, there is an urgent need for research to develop a mesenchymal stem cell therapeutic agent that can obtain a large amount of cells without damaging donor sites and has sufficient potential for treating intractable nervous system diseases.

Meanwhile, nasal inferior turbinate tissue is an independent small bone showing a shell-like shape on the lower lateral side of the nasal cavity on both the left and right sides, and is attached to the maxilla and palatine bone. The present inventors have recently reported that mesenchymal stem cells can be isolated from discarded human nasal inferior turbinate tissue and differentiated into chondrocytes, osteocytes, adipocytes, and nerve cells (KR10-1327076).

Therefore, as an alternative to overcome these problems, the present inventors have attempted to develop therapeutic agents and techniques effective for treating brain and nervous system diseases, which bypass the blood-brain barrier through minimally invasive direct brain delivery to maximize the delivery yield of a pharmaceutical composition for preventing and treating brain and nervous system diseases by administering a pharmaceutical composition for preventing and treating brain and nervous system diseases, which includes human inferior turbinate-derived mesenchymal stem cells with more accessibility compared to existing mesenchymal stem cell donor sites, into the nasal cavity including the nasal septum, olfactory mucosal epithelium, superior turbinate adjacent to the olfactory mucosal epithelium, and middle turbinate areas through the nostril or maxillary sinus.

### DISCLOSURE

### Technical Problem

The present inventors have conducted research to develop a new therapeutic agent effective for brain and nervous system diseases, and experimentally confirmed that when nasal inferior turbinate-derived mesenchymal stem cells are intranasally administered, there is an efficient therapeutic effect on brain and nervous system diseases compared to other administration routes such as intravenous administration, thereby completing the present invention.

Accordingly, an object of the present invention is to provide a pharmaceutical composition for preventing and treating brain and nervous system diseases, including cells, a drug, a biological preparation, and the like as an active ingredient, in which the pharmaceutical composition for preventing and treating brain and nervous system diseases is maximized by administering the pharmaceutical composition into the nasal cavity including the nasal septum, olfactory mucosal epithelium, superior turbinate adjacent to the olfactory mucosal epithelium, and middle turbinate areas through the nostril or maxillary sinus to bypass the blood-brain barrier through minimally invasive direct brain delivery.

Further, another object of the present invention is to provide a stem cell therapeutic agent for treating brain and nervous system diseases, including nasal inferior turbinate-derived mesenchymal stem cells as an active ingredient, in which the stem cell therapeutic agent is intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

In addition, still another object of the present invention is to provide an animal model, in which a candidate material or drug is intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

Furthermore, yet another object of the present invention is to provide a method of screening a therapeutic material for brain and nervous system diseases, the method including: (a) administering a candidate material to an animal model subject; and (b) monitoring the brain tissue of the subject, in which the candidate material is intranasally administered through the nostril or maxillary sinus,
in which the nasal cavity includes one or more areas selected from the group consisting of the nasal septum, olfactory mucosal epithelium, superior turbinate adjacent to the olfactory mucosal epithelium, and middle turbinate areas.

However, the technical problems which the present invention intends to solve are not limited to the technical problems which have been mentioned above, and other technical problems which have not been mentioned will clearly be understood by a person with ordinary skill in the art to which the present invention pertains from the following description.

### Technical Solution

To achieve the aforementioned objects of the present invention, the present invention provides a pharmaceutical composition for preventing or treating brain and nervous system diseases, including nasal inferior turbinate-derived mesenchymal stem cells as an active ingredient, in which the pharmaceutical composition is intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

As an exemplary embodiment of the present invention, the composition may be characterized by administering nasal inferior turbinate-derived mesenchymal stem cells at 5×10⁵ to 3×10⁷ cells per kg of body weight of the composition-administered subject, but is not limited thereto.

As another exemplary embodiment of the present invention, the intranasal administration may be characterized by being selected from the group consisting of local administration by injection and administration through a dispenser, but is not limited thereto.

As still another exemplary embodiment of the present invention, the administration through the dispenser may be characterized by administration through the form of an aerosol or drop delivery system, but is not limited thereto.

As yet another exemplary embodiment of the present invention, the local administration by injection may be characterized by administration through subolfactory mucosal injection, and administration into the nasal cavity including the nasal septum, olfactory mucosal epithelium, superior turbinate adjacent to the olfactory mucosal epithelium, and middle turbinate areas, but is not limited thereto.

As yet another exemplary embodiment of the present invention, the local administration by injection may be characterized by intradural injection through the skull base cribriform plate of the olfactory mucosa area, but is not limited thereto.

As yet another exemplary embodiment of the present invention, the local administration by injection may be characterized by administration through intraventricular injection through the skull base cribriform plate and subdural space, but is not limited thereto.

As yet another exemplary embodiment of the present invention, the intranasal administration may be characterized by partially removing the maxillary bone of a site to be administered and intranasal administration through this, but is not limited thereto.

As yet another exemplary embodiment of the present invention, the brain and nervous system diseases may be characterized by being selected from the group consisting of Alzheimer's disease, dementia, Parkinson's disease, stroke, cerebral ischemic stroke, intracerebral hemorrhage, Huntington's disease, multiple sclerosis, spinal cord dysfunction, traumatic brain injury, encephalitis, and degenerative brain disease, but are not limited thereto.

As yet another exemplary embodiment of the present invention, provided is a stem cell therapeutic agent for treating brain and nervous system diseases, including nasal inferior turbinate-derived mesenchymal stem cells as an active ingredient, in which the stem cell therapeutic agent is intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

Further, the present invention provides a method of preventing or treating brain and nervous system diseases, the method including administering a pharmaceutical composition including nasal inferior turbinate-derived mesenchymal stem cells as an active ingredient to a subject, in which the pharmaceutical composition is intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

In addition, the present invention provides a use of a pharmaceutical composition including nasal inferior turbinate-derived mesenchymal stem cells for preventing or treating brain and nervous system diseases, in which the pharmaceutical composition is intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

Furthermore, the present invention provides a use of inferior turbinate-derived mesenchymal stem cells for preparing a drug for preventing or treating brain and nervous system diseases, in which the inferior turbinate-derived mesenchymal stem cells are intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

Further, the present invention provides a method of screening a therapeutic material for brain and nervous system diseases, the method including: (a) administering a candidate material to an animal model subject; and (b) monitoring the brain tissue of the subject, in which the candidate material is intranasally administered through the nostril or maxillary sinus, and
the nasal cavity includes one or more areas selected from the group consisting of the nasal septum, olfactory mucosal epithelium, superior turbinate adjacent to the olfactory mucosal epithelium, and middle turbinate areas. The nasal cavity may include nasal cavities including the nasal septum or olfactory mucosal epithelium, superior turbinate adjacent to the olfactory mucosal epithelium, and middle turbinate areas, but is not limited thereto.

In addition, the present invention provides an animal model, in which a candidate material or drug is intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

Furthermore, the present invention provides a use of the animal model for screening a therapeutic material for brain and nervous system diseases.

### Advantageous Effects

The present inventors confirmed that when stem cells, and the like are intranasally injected in order to bypass the blood-brain barrier through a peri-olfactory pathway, the stem cells, and the like can be delivered to the brain through the olfactory organ and trigeminal neural pathways, various therapeutic materials including stem cells can be efficiently delivered to the brain and nervous system by the method, and side effects occurring as a result of the entire body being exposed to a drug can be reduced. In addition, intranasal administration to the nasal cavity including the nasal septum, olfactory mucosal epithelium, superior turbinate adjacent to the olfactory mucosal epithelium, and middle turbinate areas through the nostril or maxillary sinus has an excellent effect of treating brain and nervous system diseases compared to an intravenous administration method of systemic drug exposure, and an intramaxillary injection method that needs to expose the skull during cellular administration, whereas the administration method of the present invention is effective for repeated administration without the need for exposing the skull.

In addition, severe pain occurs during the existing process of acquiring mesenchymal stem cells, and a lot of time and money are consumed during the process of culturing a sufficient amount of mesenchymal stem cells, whereas the nasal inferior turbinate-derived mesenchymal stem cells of the present invention can be obtained safely and a sufficient amount of the inferior turbinate-derived mesenchymal stem cells can be obtained at a desired time, and thus the mesenchymal stem cells can be obtained at low cost with high efficiency, and it was confirmed that since the composition shares the same genetic origin with the composition-administered subject, the composition exhibits an excellent effect which is the same as or greater than the effects of bone marrow-derived mesenchymal stem cells or adipose-derived mesenchymal stem cells with fewer side effects, and thus, by intranasally administering various therapeutic materials including the nasal inferior turbinate-derived mesenchymal stem cells, the composition is expected to be capable of minimizing side effects and utilized for preventing or treating brain and nervous system diseases.

### DESCRIPTION OF DRAWINGS

FIG. 1 is a schematic view of the present invention showing that the nervous system of brain disease and anosmia patients is capable of being restored through transplantation through the nose by selecting nasal inferior turbinate-derived mesenchymal stem cells and a drug suitable for injection.

FIG. 2 is a view representing cell and drug administration locations by indicating the superior turbinate and the olfactory mucosal epithelium of the nasal septal olfactory superior region as yellow elliptical regions.

FIG. 3 is a view schematically illustrating the location of subolfactory mucosal injection administration and the drug absorption pathway through the peri-olfactory pathway after administration.

FIG. 4 is a view schematically illustrating the location of intradural injection administration and the associated drug absorption pathway.

FIG. 5 is a view schematically illustrating the location of intraventricular injection administration and the associated drug absorption pathway.

FIG. 6 is a set of views in which needles of different lengths are prepared to confirm cell and drug delivery pathways in animal experiments, and intravascular tube catheters of different lengths are also prepared together to prevent bleeding caused by damage to the olfactory mucosal epithelium.

FIG. 7 is a set of views illustrating injection into the rat's olfactory mucosal epithelium using a needle after anesthetizing the rat.

FIG. 8 is a set of views confirming the progression 1 hour after subolfactory mucosal injection of trypan blue.

FIG. 9 is a set of views confirming the progression 1 hour after subdural injection of trypan blue into a rat.

FIG. 10 is a set of views confirming the progression 3 days after injection of human nasal turbinate-derived stem cells (hNTSCs) stained with pkh26 into the rat's subdural region.

FIG. 11 is a view confirming changes in memory/learning ability by the water maze test method 6 weeks after the transplantation of PBS as a control and human nasal turbinate-derived stem cells (hNTSCs) as an experimental group into an Alzheimer's disease mouse animal model overexpressing beta-amyloid by three methods (intracranial injection, nasal dropping, and intravenous injection).

FIG. 12 is a set of views confirming human nasal turbinate-derived stem cells (hNTSCs) engrafted in the mouse brain using a human nuclei antibody by an immunostaining method 6 weeks after the transplantation of PBS as a control and human nasal turbinate-derived stem cells (hNTSCs) as an experimental group into an Alzheimer's disease mouse animal model overexpressing beta-amyloid by two methods (intracranial injection and nasal dropping).

FIG. 13 is a set of views confirming the expression of beta-amyloid and a microglial cell marker Iba-1 important for immune responses by an immunostaining method 6 weeks after the transplantation of PBS as a control and human nasal turbinate-derived stem cells (hNTSCs) as an experimental group into an Alzheimer's disease mouse animal model overexpressing beta-amyloid by three methods (intracranial injection, nasal dropping, and intravenous injection).

FIG. 14 is a set of views illustrating the results of confirming the expression of beta-amyloid and a nerve cell marker NeuN by an immunostaining method 6 weeks after transplantation of PBS as a control and human nasal turbinate-derived stem cells (hNTSCs) as an experimental group into an Alzheimer's disease mouse animal model overexpressing beta-amyloid by three methods (intracranial injection, nasal dropping, and intravenous injection).

FIG. 15 is a view showing that drugs such as cells, drugs or biological preparations can be administered by opening the external maxilla of a rabbit.

### MODES OF THE INVENTION

The present inventors confirmed that when stem cells, and the like are intranasally injected in order to bypass the blood-brain barrier through a peri-olfactory pathway, and thus the stem cells, and the like can be delivered to the brain through the olfactory organ and trigeminal neural pathways, this is a method capable of efficiently delivering various therapeutic materials including stem cells to the brain and nervous system, and side effects occurring as a result of the entire body being exposed to a drug can be reduced through this method, and confirmed that inferior turbinate-derived mesenchymal stem cells can be obtained safely and in a sufficient amount at a desired time, and thus the mesenchymal stem cells can be obtained at low cost with high efficiency, and since the composition shares the same genetic origin with the composition-administered subject, the composition exhibits an excellent effect which is the same as or greater than the effects of bone marrow-derived mesenchymal stem cells or adipose-derived mesenchymal stem cells with fewer side effects, and thus confirmed that by intranasally administering various therapeutic materials including the nasal inferior turbinate-derived mesenchymal stem cells, the composition is likely to prevent or treat brain and nervous system diseases while minimizing side effects, thereby completing the present invention.

Hereinafter, the present invention will be described in detail.

In an exemplary embodiment of the present invention, the superior turbinate and the olfactory mucosal epithelium of the nasal septal olfactory superior region are selected as the administration locations for cells and drugs, and subolfactory mucosal, subdural, and intracerebroventricular spaces were selected as intranasal local administration routes by injection (see Example 2).

In an exemplary embodiment of the present invention, it was confirmed that when trypan blue (20 μL) was injected into the olfactory mucosal epithelium of a rat, there was color up to the subdural space and a small amount of color up to the oral cavity, it was confirmed that when trypan blue (10 μL) was injected into the subdural space, the cerebellum was stained with trypan blue, and it was confirmed that when nasal inferior turbinate-derived mesenchymal stem cells (10×10⁵/10 μL) were stained with pkh26(red) and injected into the subdural space, the stained nasal inferior turbinate-derived mesenchymal stem cells were present in the cerebellum (see Example 3).

In another exemplary embodiment of the present invention, it was confirmed that intranasal administration in an Alzheimer's mouse animal model has a much better recovery effect on memory/learning ability in mice than intravenous administration (see Example 4-1).

In still another embodiment of the present invention, it was confirmed that the survival of human nasal turbinate-derived-stem cells (hNTSCs) was not confirmed in the brain when administered intravenously in an Alzheimer's mouse animal model, whereas nasal inferior turbinate-derived stem cells were confirmed to have survived well when intranasally administered (see Example 4-2).

In yet another embodiment of the present invention, it was confirmed that intranasal administration in an Alzheimer's mouse animal model showed a greater decrease in the expression of beta-amyloid than intravenous administration (see Example 4-3).

In yet another exemplary embodiment of the present invention, it was confirmed that beta-amyloid expression was decreased and the expression of nerve cells was significantly increased during intranasal administration in an Alzheimer's mouse animal model (see Example 4-4).

Therefore, the present invention may provide a pharmaceutical composition for preventing or treating brain and nervous system diseases, including nasal inferior turbinate-derived mesenchymal stem cells as an active ingredient, and a stem cell therapeutic agent for treating brain and nervous system diseases.

As used herein, the term “stem cell” refers to, as a cell that is the basis of a cell or tissue that constitutes a subject, a cell that may be repeatedly divided to achieve self-renewal and has a multi-differentiation ability to differentiate into cells having specific functions according to the environment. Stem cells are generated in all tissues during the fetal development process, and are found in some tissues where cells are actively replaced, such as bone marrow and epithelial tissues, even in adults. Stem cells are classified into totipotent stem cells that are formed when fertilized eggs begin to divide, pluripotent stem cells that are located in the inner cell mass of the blastocyst that is created by the continuous division of these cells, and multipotent stem cells present in mature tissues and organs. In this case, multipotent stem cells are cells that can differentiate only into cells specific for tissues and organs in which these cells are included, and are involved in not only the growth and development of each tissue and organ in the fetal period, neonatal period, and adult period, but also functions of maintaining the homeostasis of living tissue and inducing regeneration during tissue damage. Such tissue-specific multipotent cells are collectively referred to as adult stem cells.

Mesenchymal stem cells classified as adult stem cells are cells that have drawn attention as a material for regenerative medicine, may be collected from tissues such as bone marrow, cord blood, and umbilical cord blood, and have the ability to differentiate into cells constituting various human body tissues such as adipose tissue cells, osteocytes, chondrocytes, nerve cells, and cardiomyocytes unlike blood stem cells. In the present invention, mesenchymal stem cells separated from human inferior turbinate tissue were used.

Among adult mesenchymal stem cells, bone marrow-derived mesenchymal stem cells and adipose tissue-derived mesenchymal stem cells have disadvantages in that surgery to obtain the mesenchymal stem cells is accompanied by severe pain and is time-consuming, an amount of obtained mesenchymal stem cells is very small, much time and money is spent in the process of culturing a clinically sufficient amount, and the risk of infection and cell loss is high. In addition, cord blood-derived mesenchymal stem cells have a problem in that it is difficult to obtain the mesenchymal stem cells at the required time and need to be stored for a long period of time.

In contrast, human inferior turbinate-derived mesenchymal stem cells have advantages in that surgery to obtain the stem cells is accompanied by very little bleeding and pain and takes less time, mesenchymal stem cells can be continuously secured through the recycling of mesenchymal stem cells isolated from discarded inferior turbinate tissue during inferior turbinate surgery (rhinitis surgery) most frequently performed in the otolaryngology field, and the ability of stem cells to proliferate is higher than that of the bone marrow-derived and adipose tissue-derived mesenchymal stem cells.

As used herein, the term “cell therapeutic agent” refers to a drug used for the purpose of treatment, diagnosis, and prevention, by using a cell or tissue prepared through isolation from a human, culture and specific manipulation, and specifically, it refers to a drug used for the purpose of treatment, diagnosis, and prevention through a series of actions of in vitro multiplying and sorting allogenic or xenogenic cells or changing the biological characteristics of cells by other methods for the purpose of recovering the functions of cells and tissues. Cell therapeutic agents are largely classified into somatic cell therapeutic agents and stem cell therapeutic agents according to the degree of cell differentiation, and the present invention particularly relates to a stem cell therapeutic agent.

As used herein, the term “brain and nervous system diseases” is characterized in that the death or degeneration of specific brain cells progresses temporarily or over a long period of time, but once brain cells are dead, they are not regenerated, thereby leading to the ultimate fatal loss of brain function. In particular, brain dysfunction accompanied by progressive deterioration in cognitive, sensory, motor, and systemic functions is eventually a disease which leads to changes in personality and behavior, and patients being unable to take care of themselves, and is characterized by being selected from the group consisting of Alzheimer's disease, dementia, Parkinson's disease, stroke, cerebral ischemic stroke, intracerebral hemorrhage, Huntington's disease, multiple sclerosis, spinal cord dysfunction, traumatic brain injury, encephalitis, and degenerative brain disease, but is not limited thereto.

The pharmaceutical composition of the present invention may further include an appropriate carrier, an appropriate excipient, and an appropriate diluent, which are typically used to prepare a pharmaceutical composition. The excipient may be, for example, one or more selected from the group consisting of a diluent, a binder, a disintegrant, a lubricant, an adsorbent, a moisturizer, a film-coating material, and a controlled release additive.

The pharmaceutical composition according to the present invention may be used by being formulated into the form of a powder, a granule, a sustained-release granule, an enteric granule, a liquid, a collyrium, an elixir, an emulsion, a suspension, a spirit, a troche, aromatic water, a limonade, a tablet, a sustained-release tablet, an enteric tablet, a sublingual tablet, a hard capsule, a soft capsule, a sustained-release capsule, an enteric capsule, a pill, a tincture, a soft extract agent, a dry extract agent, a fluid extract agent, an injection, a capsule, a perfusate, an external preparation such as a plaster, a lotion, a paste, a spray, an inhalant, a patch, a sterilized injection solution, or an aerosol, and the external preparation may have a formulation such as a cream, a gel, a patch, a spray, an ointment, a plaster, a lotion, a liniment, a paste or a cataplasma.

Examples of a carrier, an excipient or a diluent which may be included in the composition according to the present invention include lactose, dextrose, sucrose, an oligosaccharide, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxy benzoate, propyl hydroxybenzoate, talc, magnesium stearate, and mineral oil.

When the pharmaceutical composition is prepared, the pharmaceutical composition is prepared using a diluent or excipient, such as a filler, an extender, a binder, a wetting agent, a disintegrant, and a surfactant, which are commonly used.

As an additive of the tablet, powder, granule, capsule, pill, and troche according to the present invention, it is possible to use an excipient such as corn starch, potato starch, wheat starch, lactose, white sugar, glucose, fructose, D-mannitol, precipitated calcium carbonate, synthetic aluminum silicate, calcium monohydrogen phosphate, calcium sulfate, sodium chloride, sodium hydrogen carbonate, purified lanolin, microcrystalline cellulose, dextrin, sodium alginate, methyl cellulose, carboxymethyl cellulose sodium, kaolin, urea, colloidal silica gel, hydroxypropyl starch, hydroxypropyl methylcellulose (HPMC) 1928, HPMC 2208, HPMC 2906, HPMC 2910, propylene glycol, casein, calcium lactate, and Primojel; and a binder such as gelatin, arabic gum, ethanol, agar powder, cellulose acetate phthalate, carboxymethyl cellulose, carboxymethyl cellulose calcium, glucose, purified water, sodium caseinate, glycerin, stearic acid, carboxymethyl cellulose sodium, methylcellulose sodium, methylcellulose, microcrystalline cellulose, dextrin, hydroxycellulose, hydroxypropyl starch, hydroxymethyl cellulose, purified shellac, starch, hydroxypropyl cellulose, hydroxypropyl methyl cellulose, polyvinyl alcohol, and polyvinylpyrrolidone, and it is possible to use a disintegrant such as hydroxypropyl methyl cellulose, corn starch, agar powder, methyl cellulose, bentonite, hydroxypropyl starch, carboxymethyl cellulose sodium, sodium alginate, carboxymethyl cellulose calcium, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, arabic gum, amylopectin, pectin, sodium polyphosphate, ethyl cellulose, white sugar, magnesium aluminum silicate, a D-sorbitol solution, and light anhydrous silicic acid; and a lubricant such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol 4000 and 6000, liquid paraffin, hydrogenated soybean oil (Lubriwax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid.

As an additive for liquid formulation according to the present invention, it is possible to use water, diluted hydrochloric acid, diluted sulfuric acid, sodium citrate, sucrose monostearate, polyoxyethylene sorbitol fatty acid esters (Tween esters), polyoxyethylene monoalkyl ethers, lanolin ether, lanolin ester, acetic acid, hydrochloric acid, aqueous ammonia, ammonium carbonate, potassium hydroxide, sodium hydroxide, prolamin, polyvinyl pyrrolidone, ethyl cellulose, carboxymethyl cellulose sodium, and the like.

In a syrup according to the present invention, a solution of sucrose, other sugars or sweeteners, and the like may be used, and a fragrance, a colorant, a preservative, a stabilizer, a suspending agent, an emulsifier, a thickener, and the like may be used, if necessary.

Purified water may be used for the emulsion according to the present invention, and an emulsifier, a preservative, a stabilizer, a fragrance, and the like may be used, if necessary.

In the suspending agent according to the present invention, a suspending agent such as acacia, tragacanth, methyl cellulose, carboxymethyl cellulose, carboxymethyl cellulose sodium, microcrystalline cellulose, sodium alginate, hydroxypropyl methyl cellulose, HPMC 1828, HPMC 2906, and HPMC 2910 may be used, and a surfactant, a preservative, a colorant, and a fragrance may be used, if necessary.

The injection according to the present invention may include: a solvent such as distilled water for injection, 0.9% sodium chloride injection, Ringer's injection, dextrose injection, dextrose+sodium chloride injection, PEG, lactated Ringer's injection, ethanol, propylene glycol, non-volatile oil-sesame oil, cottonseed oil, peanut oil, corn oil, ethyl oleate, isopropyl myristate, and benzoic acid benzene; a solubilizing agent such as sodium benzoate, sodium salicylate, sodium acetate, urea, urethane, monoethyl acetamide, butazolidin, propylene glycol, Tweens, nijungtinateamide, hexamine, and dimethylacetamide; a buffer such as a weak acid or a salt thereof (acetic acid and sodium acetate), a weak base and a salt thereof (ammonia and ammonium acetate), an organic compound, a protein, albumin, peptone, and gums: an isotonic agent such as sodium chloride; a stabilizer such as sodium bisulfite (NaHSO₃), carbon dioxide gas, sodium metabisulfite (Na₂S₂O₃), sodium sulfite (Na₂SO₃), nitrogen gas (N₂), and ethylenediaminetetraacetic acid: a sulfating agent such as 0.1% sodium bisulfide, sodium formaldehyde sulfoxylate, thiourea, disodium ethylenediaminetetraacetate, and acetone sodium bisulfite: an analgesic such as benzyl alcohol, chlorobutanol, procaine hydrochloride, glucose, and calcium gluconate; and a suspending agent such as carboxymethyl cellulose sodium, sodium alginate, Tween 80, and aluminum monostearate.

In a suppository according to the present invention, it is possible to use a base such as cacao butter, lanolin, Witepsol, polyethylene glycol, glycerogelatin, methylcellulose, carboxymethyl cellulose, a mixture of stearic acid and oleic acid, Subanal, cottonseed oil, peanut oil, palm oil, cacao butter+cholesterol, lecithin, ranetwax, glycerol monostearate, Tween or Span, Imhausen, monolen (propylene glycol monostearate), glycerin, Adeps solidus, Buytyrum Tego-G, Cebes Pharma 16, hexalide base 95, Cotomar, Hydroxote SP, S-70-XXA, S-70-XX75(S-70-XX95), Hydrokote 25, Hydrokote 711, idropostal, Massa estrarium (A, AS, B, C, D, E, I, T), Massa-MF, Masupol, Masupol-15, Neosupostal-ene, Paramound-B, Suposhiro (OSI, OSIX, A, B, C, D, H, L), suppository base IV types (AB, B, A, BC, BBG, E, BGF, C, D, 299), Supostal (N, Es), Wecobee (W, R, S, M,Fs), and Tegester triglyceride bases (TG-95, MA, 57).

A solid formulation for oral administration includes a tablet, a pill, a powder, a granule, a capsule, and the like, and the solid formulation is prepared by mixing at least one excipient, for example, starch, calcium carbonate, sucrose or lactose, gelatin, and the like with an extract. Further, in addition to a simple excipient, lubricants such as magnesium stearate and talc are also used.

A liquid formulation for oral administration corresponds to a suspension, a liquid for internal use, an emulsion, a syrup, and the like, and the liquid formulation may include, in addition to water and liquid paraffin which are simple commonly used diluents, various excipients, for example, a wetting agent, a sweetener, a fragrance, a preservative, and the like. Examples of a formulation for parenteral administration include an aqueous sterile solution, a non-aqueous solvent, a suspension, an emulsion, a freeze-dried preparation, and a suppository. As the non-aqueous solvent and the suspension, it is possible to use propylene glycol, polyethylene glycol, a vegetable oil such as olive oil, an injectable ester such as ethyl oleate, and the like.

The pharmaceutical composition according to the present invention is administered in a pharmaceutically effective amount. The term “pharmaceutically effective amount” as used herein refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and other factors well known in the medical field, and is preferably characterized by administering nasal inferior turbinate-derived mesenchymal stem cells at 5×10⁵ to 3×10⁷ cells per kg of the composition-administered subject's body weight, but is not limited thereto.

The pharmaceutical composition according to the present invention may be preferably administered simultaneously, separately or sequentially with a drug to be used in combination, and may be administered once or multiple times, in order to enhance the therapeutic effect. It is important to administer the composition in a minimum amount that can obtain the maximum effect without any side effects, in consideration of all the aforementioned factors, and this amount may be easily determined by those skilled in the art. Specifically, the effective amount of the pharmaceutical composition according to the present invention may vary depending on the age, sex, condition, and body weight of a patient, the absorption rate, inactivation rate and excretion rate of the active ingredient in vivo, the type of disease, and the drug to be used in combination.

The intranasal administration is characterized by being selected from the group consisting of local administration by injection and administration through a dispenser, but is not limited thereto.

As used herein, the term “dispenser” means that a strictly defined amount of drug can be administered directly to the nose, and the drug can be administered through the form of an aerosol or drop delivery system, but is not limited thereto.

The “local administration by injection” may administer the drug through subolfactory mucosal injection, intradural injection through the cribriform plate of the skull base, or intraventricular injection through the cribriform plate and the subdural space, but is not limited thereto.

As used herein, the term “olfactory mucosa” refers to a region in the nose which has receptors for the sense of smell.

As used herein, the term “skull base” refers to a general term for an area which constitutes the lower part of the cranial cavity in the skull, and an area seen from the inside (upper) surface refers to the upper surface of the skull base, and an area seen from the outside (lower) surface refers to the lower surface of the skull base.

As used herein, the term “cribriform plate” refers to the skeletal structure at the rostral base of the brain, and the olfactory mucosa where the cell bodies of the olfactory receptors are located is arranged along with the cribriform plate.

As used herein, the term “maxillary sinus” refers to the paranasal sinuses under the eyes, and a thin septum separates the anterior and posterior maxillary sinuses.

As used herein, the term “nasal septum” refers to a structure which is mainly composed of cartilage and osseous lamina, supports the bridge and tip of the nose and is covered with a mucous membrane as a partition wall that divides the inside of the nose (nasal cavity) into left and right parts, and has a form that supports the bridge of the nose (nasal dorsum) and the tip of the nose (nasal tip) because the nose septal cartilage (nasal septal cartilage) is located anteriorly, and the ethmoid bone perpendicular plate (perpendicular plate of ethmoid bone), the vomer bone (vomer), the crest of maxillary bone (maxillary crest), and the crest of palatine bone (palatal crest) are located posteriorly.

As used herein, the term “superior turbinate” refers to a small shell-like bony protuberance protruding from the upper side to the lower side of the outer wall of the nasal cavity or an area covered with its mucous membrane. This is not an independent bone, but the lower side forms the superior meatus between the superior turbinate and the middle turbinate. Further, there may be a small nasal superior turbinate on the upper side behind the superior meatus.

As used herein, the term “middle turbinate” refers to a shell-like bony protuberance protruding downward from approximately the center of the outer wall of the nasal cavity and a portion covered with mucous membrane. The upper side thereof forms the superior meatus between the middle turbinate and the superior turbinate, and the lower side thereof forms the middle meatus between the middle turbinate and the inferior turbinate. In addition, this bone is not an independent piece, but is a part of the ethmoid bone.

As used herein, the term “brain dura mater” refers to the outermost, tough, and connective tissue membrane in the meninges, the brain dura mater is made up of two layers, the outer layer is a layer to which original bone has migrated and has many blood vessels and nerves, and the inner layer is the intrinsic dura mater and is a dense connective tissue containing elastic fibers.

As used herein, the term “ventricle” refers to a space within the human brain, and is surrounded by the ventricular membrane. There are three ventricles, the lateral ventricle, the third ventricle, and the fourth ventricle, all of which are continuous spaces, the inside of the ventricles is filled with a fluid called cerebrospinal fluid, and a certain amount of this fluid is produced every day and circulated in the ventricular system while being decomposed.

As used herein, the term “intranasal administration” may be characterized by partially removing the maxillary bone of a site to be administered and administration, but is not limited thereto. Specifically, the intranasal administration may be local administration in the brain through the olfactory bulb region observed after opening the maxillary bone area of a subject, and then administration by the process of reclosing and suturing the maxillary bone.

The pharmaceutical composition of the present invention is determined by the type of drug that is an active ingredient, as well as various related factors such as the disease to be treated, the route of administration, the age, sex, and body weight of a patient, and the severity of the disease. Specifically, the effective amount of the composition according to the present invention may vary depending on the patient's age, sex, and body weight, and generally, 0.001 to 150 mg of the composition and preferably, 0.01 to 100 mg of the composition, per 1 kg of body weight, may be administered daily or every other day or administered once to three times a day. However, since the effective amount may be increased or decreased depending on the administration route, the severity of obesity, gender, body weight, age, and the like, the dosage is not intended to limit the scope of the present invention in any way.

As used herein, the “subject” refers to a subject in need of treatment of a disease, and more specifically, may be a mammal such as a human or a non-human primate, a mouse, a rat, a dog, a cat, a horse, and a cow, but is not limited thereto.

As used herein, the term “administration” refers to the provision of a predetermined composition of the present invention to a subject thereof by any suitable method.

As used herein, the “prevention” refers to all actions that suppress or delay the onset of a target disease, and the “treatment” refers to all actions that ameliorate or beneficially change a target disease and the resulting metabolic abnormalities by administration of the pharmaceutical composition according to the present invention, and the “amelioration” refers to all actions that reduce a target disease and associated parameters, for example, the severity of symptoms, by administration of the composition according to the present invention.

Furthermore, the present invention provides a method of screening a therapeutic material for brain and nervous system diseases, the method including: (a) administering a candidate material to an animal model subject; and (b) monitoring the brain tissue of the subject, in which the candidate material is intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

The term “candidate material” used in reference to the screening method of the present invention refers to an unknown material that is used in screening to test whether it affects the brain and nervous system diseases of the present invention. The candidate material includes small interference RNA (siRNA), short hairpin RNA (shRNA), microRNA (miRNA), ribozymes, DNAzymes, peptide nucleic acids (PNAs), antisense oligonucleotides, antibodies, aptamers, natural extracts or chemicals, but is not limited thereto.

Further, the cells used in Step (a) may be provided in the form of experimental animals, and contact with the candidate material is characterized by intranasal administration or intramaxillary administration.

Monitoring in Step (b) of the present invention may include the measurement of an mRNA expression level or the measurement of a protein expression level.

In Step (b) of the present invention, the measurement of an mRNA expression level may be a measurement using one or more methods selected from the group consisting of RT-PCR, quantitative or semi-quantitative RT-PCR, quantitative or semi-quantitative real-time RT-PCR, northern blot and DNA or RNA chips, but is not limited thereto.

In Step (b) of the present invention, the measurement of a protein expression level may be measurement using one or more methods selected from the group consisting of western blot, ELISA, radioimmunoassay, radioimmunodiffusion, Ouchterlony immunodiffusion, rocket immunoelectrophoresis, immunohistochemical staining, immunoprecipitation analysis, complement fixation analysis, FACS and protein chips, but is not limited thereto.

In the present invention, a control may be an animal model that is not treated with a candidate material, but is not limited thereto.

In the present specification, a brain tissue to be monitored may be preferably intracranial biological tissue, brain tissue, brain cells, or cerebral blood vessels, preferably cerebral cells, cerebral tissue, or cerebral blood vessels, and more preferably cerebral cortex, hippocampus or neural tissue, but is not limited thereto.

In addition, the present invention provides an animal model, in which a candidate material or drug is intranasally administered through the nostril or maxillary sinus, and the nasal cavity includes one or more areas selected from the group consisting of the nasal septum, olfactory mucosal epithelium, superior turbinate adjacent to the olfactory mucosal epithelium, and middle turbinate areas.

In the present invention, the term “candidate material” is as described above.

In the present invention, the term “drug” includes small interference RNA (siRNA), short hairpin RNA (shRNA), microRNA (miRNA), ribozymes, DNAzymes, peptide nucleic acids (PNAs), antisense oligonucleotides, antibodies, aptamers, cells, natural extracts or chemicals, but is not limited thereto.

The cells include stem cells, but are not limited thereto.

The animal model may be prepared using mammals other than humans, and the mammals other than humans may be monkeys, rats, mice, rabbits, dogs, primates, and the like, preferably Muridae animals, but are not limited thereto.

Throughout the specification of the present application, when one part “includes” one constituent element, unless otherwise specifically described, this does not mean that another constituent element is excluded, but means that another constituent element may be further included. Throughout the specification of the present application, a term of a degree, such as “about” or “substantially”, is used in a corresponding numerical value or used as a meaning close to the numerical value when natural manufacturing and material tolerance errors are presented in a described meaning, and is used to prevent an unconscientious infringer from illegally using disclosed contents including a numerical value illustrated as being accurate or absolute in order to help understanding of the present invention. Throughout the specification of the present application, a term of a degree, such as a “step . . . ” or a “step of . . . ” does not mean a “step for”.

Throughout the specification of the present application, the term “combination(s) thereof” included in the Markush type expression means a mixture or combination of at least one selected from the group consisting of constituent elements described in the Markush type expression, and means including at least one selected from the group consisting of the constituent elements.

Throughout the specification of the present application, the description “A and/or B” means “A or B, or A and B”.

Specific steps may be performed out of the order described where certain embodiments can be implemented differently. For example, two steps described in succession may be performed substantially concurrently, or may be performed in the reverse order to that described.

### MODE FOR INVENTION

Hereinafter, preferred examples for helping with understanding of the present invention will be suggested. However, the following examples are provided only so that the present invention may be more easily understood, and the content of the present invention is not limited by the following examples.

### EXAMPLES

### Example 1. Isolation and Culture of Human Inferior Turbinate-Derived Mesenchymal Stem Cells (hNTSCs)

With the consent of a patient before surgery, an inferior turbinate tissue was collected during inferior turbinectomy, and immediately after the inferior turbinate tissue was collected, the tissue was washed with physiological saline containing gentamicin (Kukje Pharma. CO. LTD., Seongnam, Korea) three to five times.

In order to isolate human inferior turbinate-derived mesenchymal stem cells from the inferior turbinate tissue collected by the above process, the collected tissue was first stored in refrigerator at 4° C. and then washed three times with an antibiotic-antifungal solution (Gibco, Gaithersberg, MD) at room temperature. Then, the tissue was again washed twice with neutral phosphate buffered saline (PBS), and then finely cut into a size of 0.5 mm³ using surgical scissors.

The cut tissue was placed on a 100-mm culture dish, covered with a sterilized slide glass, adhered to the culture dish, and cultured in an incubator under an environment of 37° C. and 5% CO₂ by adding Dulbecco's Modified Eagle's Media (DMEM) supplemented with 10% fetal bovine serum (FBS) thereto. After the tissue was cultured for 2 to 3 weeks, the slide glass was removed, cells floating in the culture solution were washed and discarded, human inferior turbinate-derived mesenchymal stem cells attached to the bottom of the culture dish were detached from the bottom, and cells that were subcultured up to the third generation were used.

### Example 2. Intranasal Local Administration Route by Injection

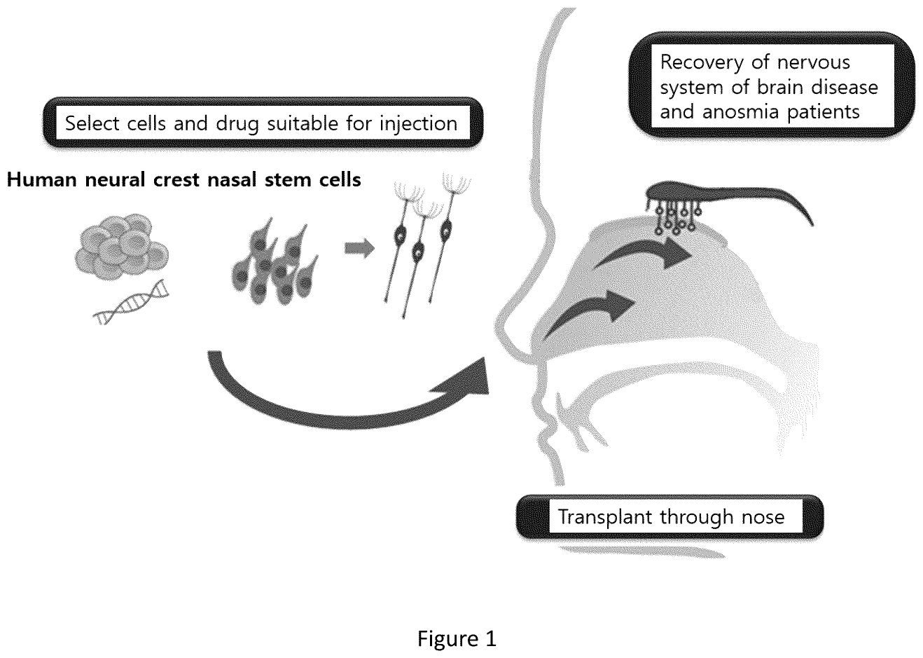

As illustrated in FIG. 1, the present inventors isolated and cultured human inferior turbinate-derived mesenchymal stem cells, as shown in Example 1, in order to restore the nervous system more quickly and efficiently, and then locally injected the isolated and cultured human inferior turbinate-derived mesenchymal stem cells and a drug through the nose.

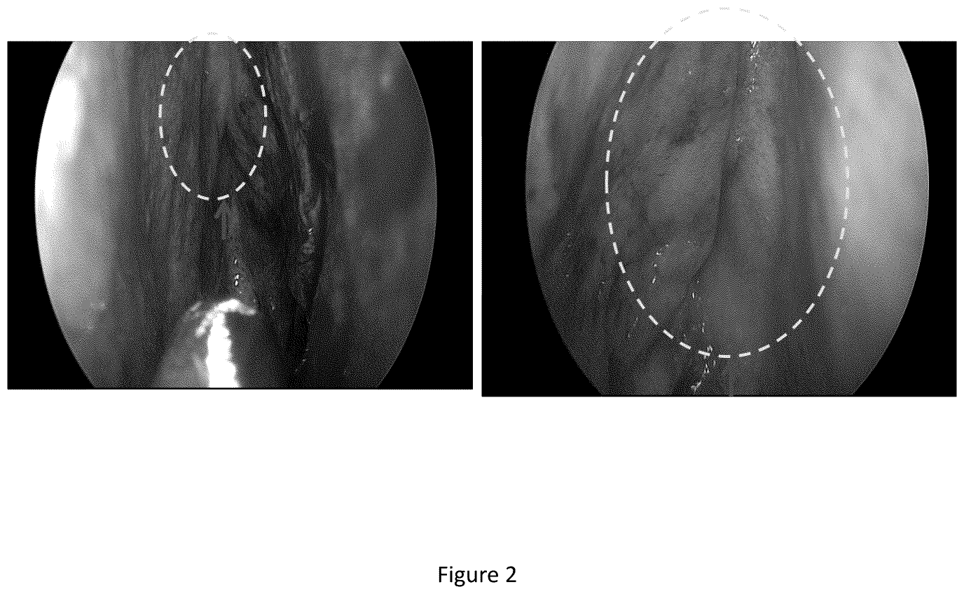

As illustrated in FIG. 2, the olfactory mucosal epithelium of the superior turbinate and the posterior upper part of the nasal septum indicated as yellow elliptical regions, were selected as cell and drug administration locations.

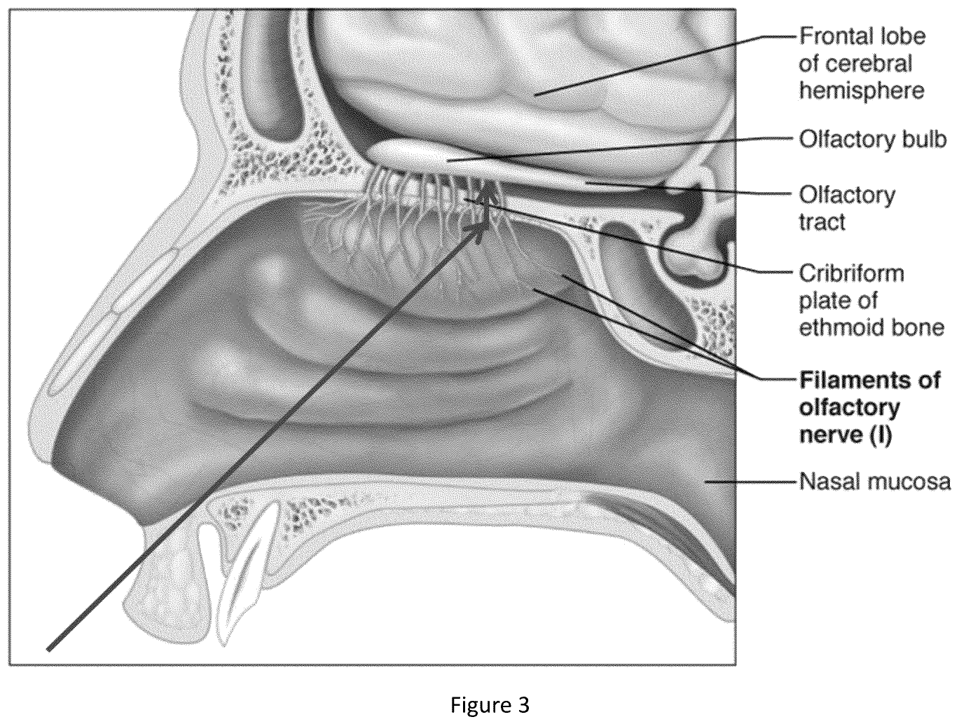

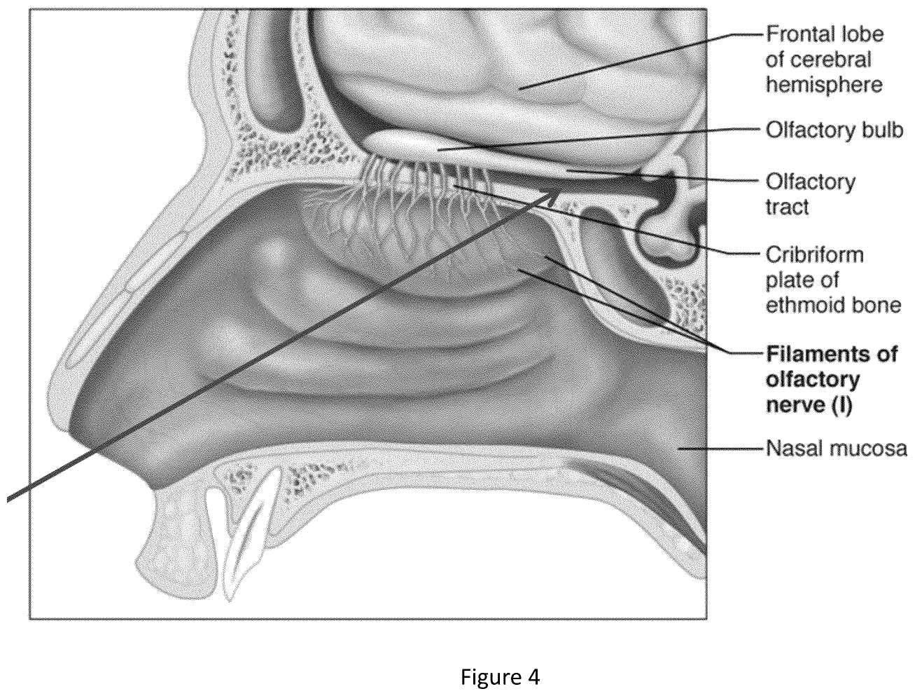

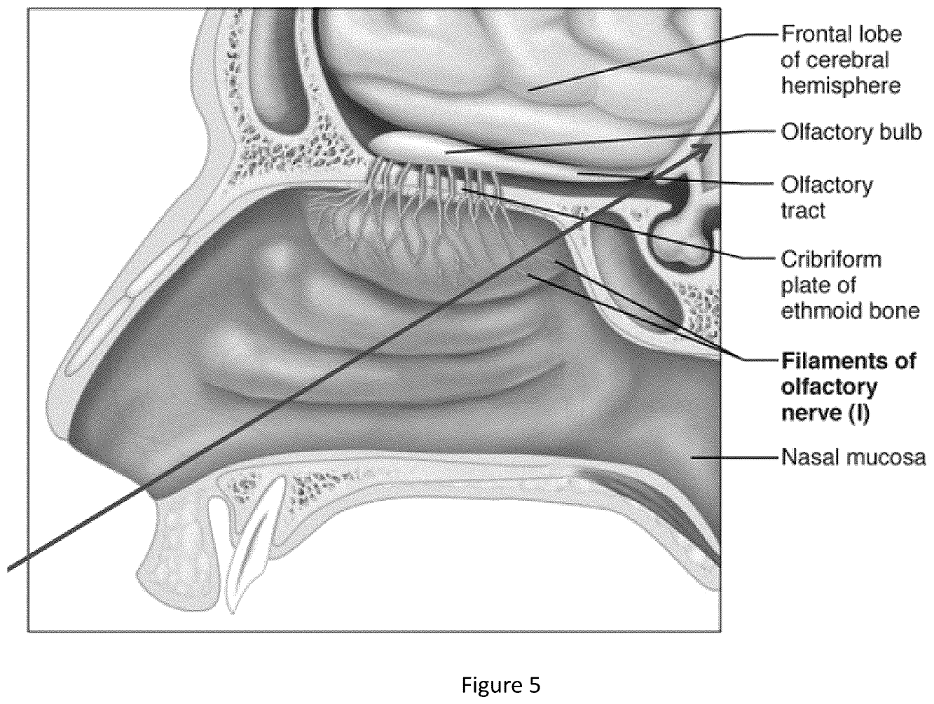

As a specific route of local administration through the regions indicated above, there are a route by which the drug is absorbed through the olfactory perineural pathway by administration through subolfactory mucosal injection as illustrated in FIG. 3, a route by which the drug is absorbed by administration through intraventricular injection through the skull base cribriform plate as illustrated in FIG. 4, and a route by which the drug is absorbed by administration through intraventricular injection through the skull base cribriform plate and subdural space as illustrated in FIG. 5.

### Example 3. Preparation and Results of Rat Experiments to Confirm Cell and Drug Delivery Routes

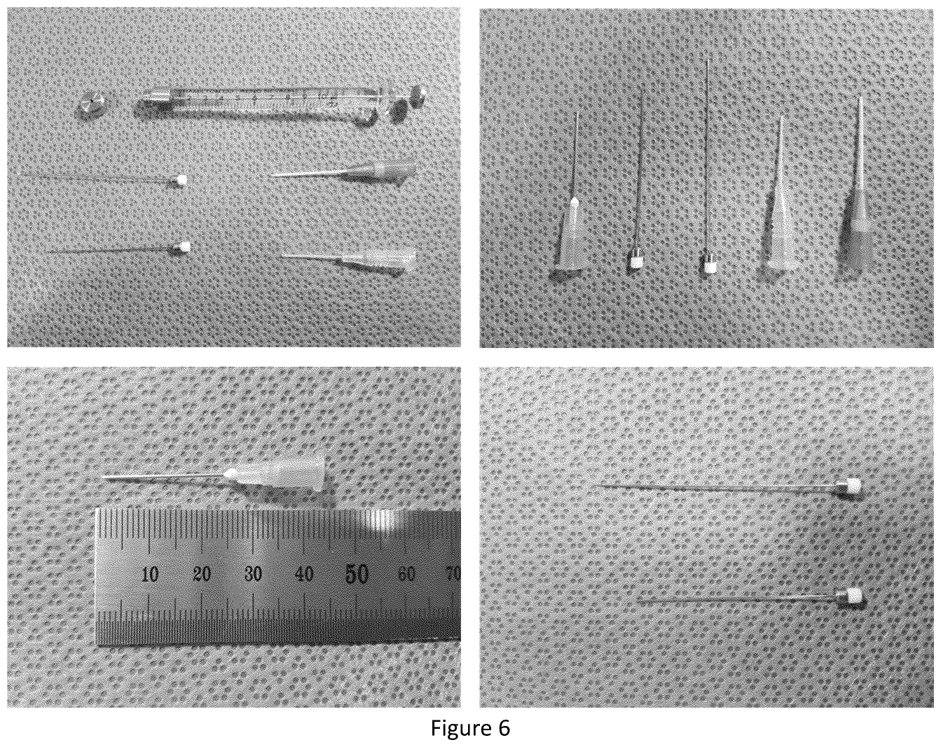

As shown in Example 2, to deliver cells and drugs to the three local administration routes, since the length from the nostril to the olfactory mucosal epithelium, brain dura mater, and ventricle varied slightly from rat to rat, needles of different lengths were prepared as illustrated in FIG. 6. Further, intravascular tube catheters of different lengths were also prepared together to prevent bleeding caused by damage to the olfactory mucosal epithelium of the rat.

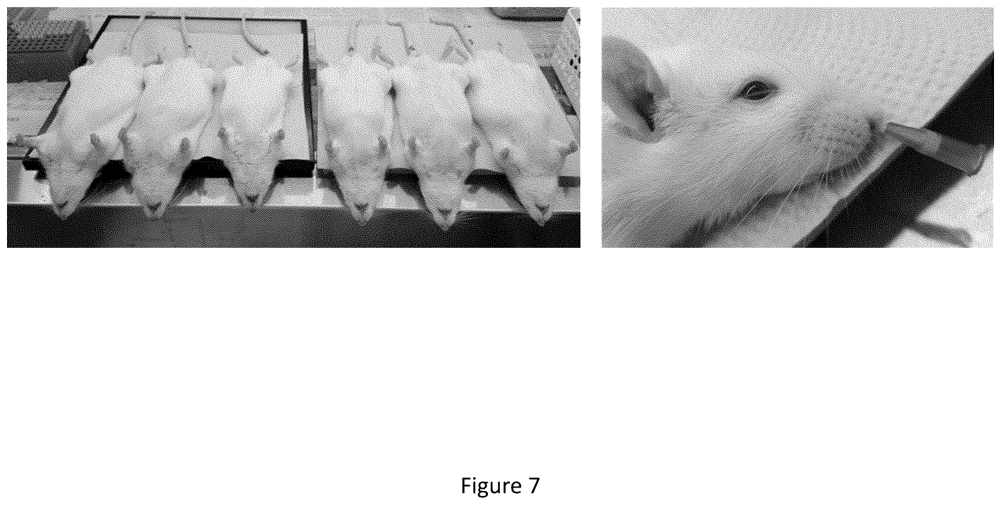

Thereafter, as illustrated in FIG. 7, after the rat was anesthetized and then laid on its side, a catheter that matches the length to the olfactory mucosal epithelium of each rat was selected and fixed to a portion to be injected, and then injection was made using a needle that matches each length.

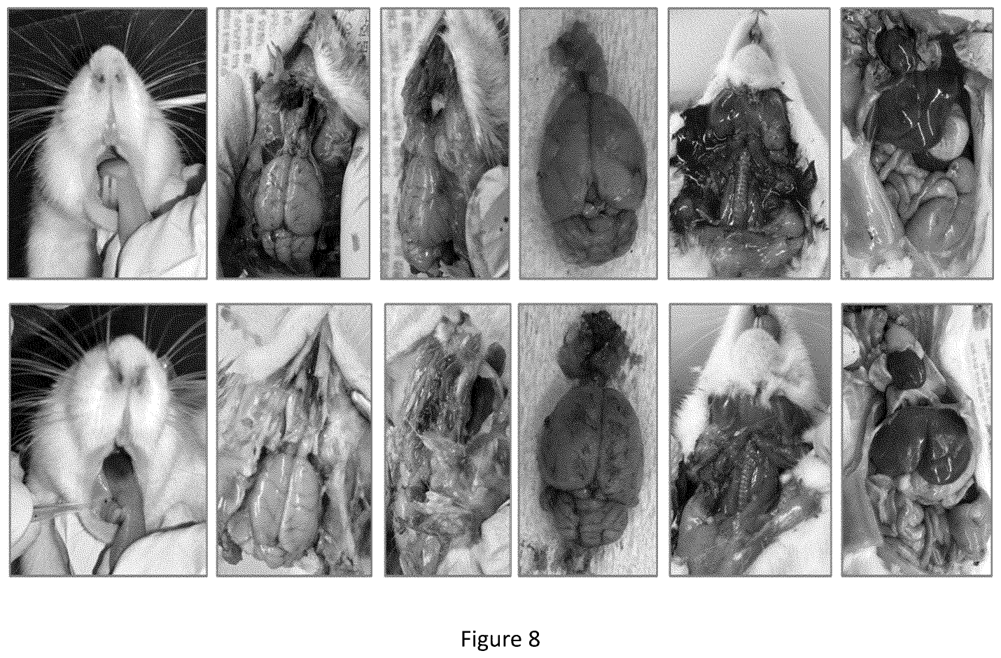

As a result, as illustrated in FIG. 8, as a result of injecting trypan blue (20 μL) into the olfactory mucosal epithelium of the rat and then observing the parts around the administration site 1 hour later, there was color up to the brain subdural space, and a small amount of color up to the oral cavity, but migration from the respiratory tract (trachea) to other organs could not be confirmed.

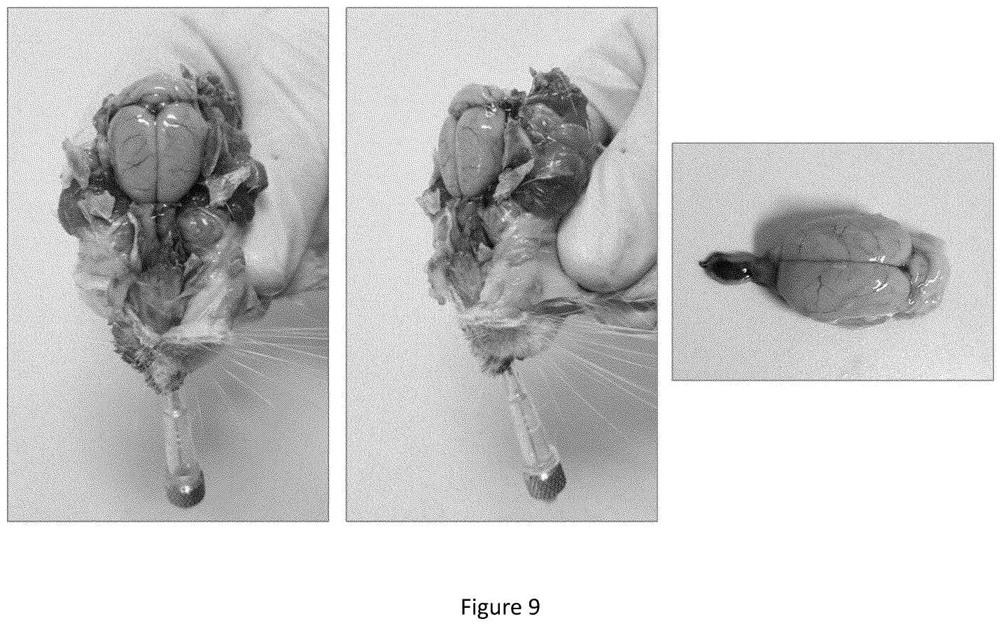

In addition, as illustrated in FIG. 9, after trypan blue (10 μL) was injected into the brain subdural space, the possibility of a delivery route to the cerebellum was identified by confirming that the cerebellum was stained with trypan blue one hour later.

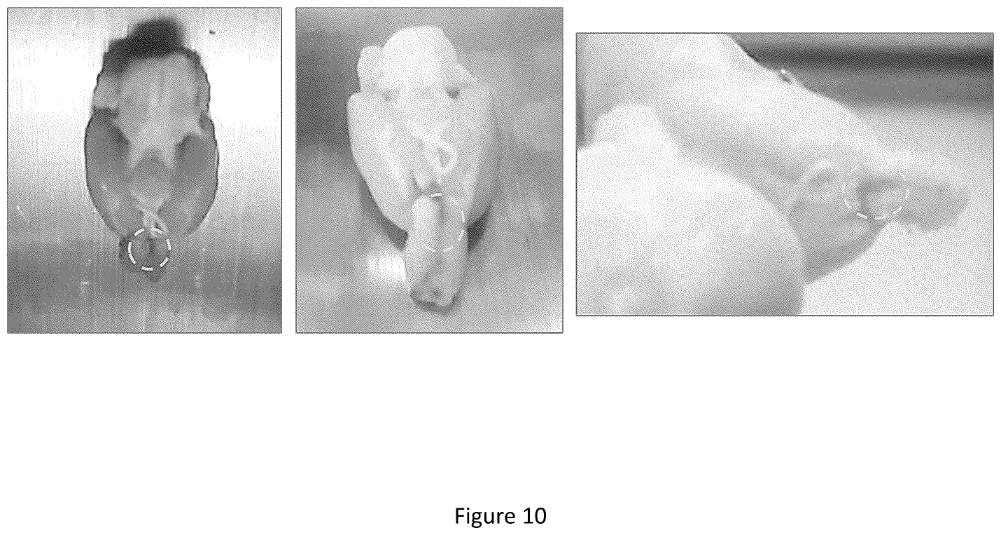

Furthermore, as illustrated in FIG. 10, after human inferior turbinate-derived mesenchymal stem cells (1*10⁵/10 μL) were stained with pkh26 (red) and injected into the brain dura mater, it was confirmed that the stained human inferior turbinate-derived mesenchymal stem cells were present in the cerebellum when the cerebellum was observed by sacrificing the rats.

That is, it can be seen that when cells and drugs are locally administered by injection through the route shown in Example 2, cells and drugs can be efficiently delivered to the brain subdural space or cerebellum.

### Example 4. Confirmation of Effect of Intranasal Administration in Beta-Amyloid-Overexpressing Alzheimer's Mouse Animal Model

### Example 4-1. Confirmation of Changes in Memory/Learning Ability by Intranasal Administration of Inferior Turbinate-Derived Stem Cells (hNTSCs)

Changes in memory/learning ability were observed by the water maze test method 6 weeks after the transplantation of PBS as a control and human inferior turbinate-derived stem cells (hNTSCs) as an experimental group into an Alzheimer's disease mouse animal model overexpressing beta-amyloid by three methods (intracranial injection, IC; nasal dropping, IN; and intravenous injection, IV).

5×FAD gene-overexpressing mice (transgenic mice) were used as an Alzheimer's disease mouse animal model. 5×FAD-transgenic mice overexpress human amyloid beta (A4) precursor protein 695 (APP) along with human presenilin 1 phase Swedish (K670N, M671L), Florida (1716V), and London (V717I) Familial Alzheimer's Disease (FAD) mutations. In the mouse animal model, the amyloid precursor protein, which is a precursor of beta-amyloid protein, and the presenilin-1 enzyme, which decomposes this protein to play an important role in beta-amyloid protein synthesis, are overexpressed to exhibit symptoms of Alzheimer's disease.

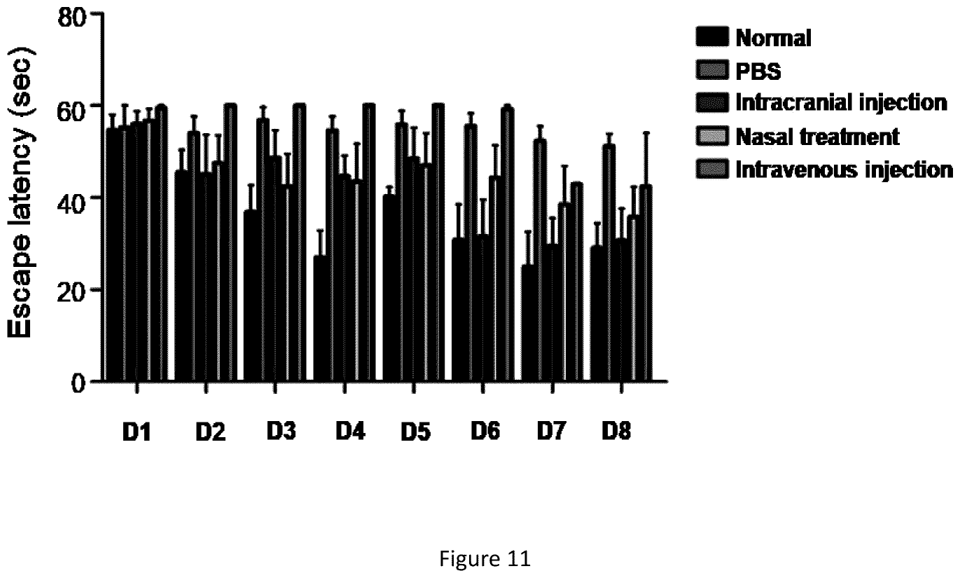

As a result, as illustrated in FIG. 11, it was confirmed that the memory/learning ability was recovered more rapidly in the stem cell-transplanted group than in the PBS-injected group, and the group in which the cells were transplanted by the IN method exhibited a higher therapeutic effect.

### Example 4-2. Confirmation of Changes in Memory/Learning Ability by Intranasal Administration of Inferior Turbinate-Derived Stem Cells (hNTSCs)

In an Alzheimer's disease mouse animal model overexpressing beta-amyloid, hNTSCs engrafted in the mouse brain were confirmed using a human nuclei antibody by an immunostaining method 6 weeks after the transplantation of PBS as a control and human inferior turbinate-derived stem cells (hNTSC) as an experimental group by three methods (intracranial injection; IC, nasal dropping; IN, and intravenous injection: IV).

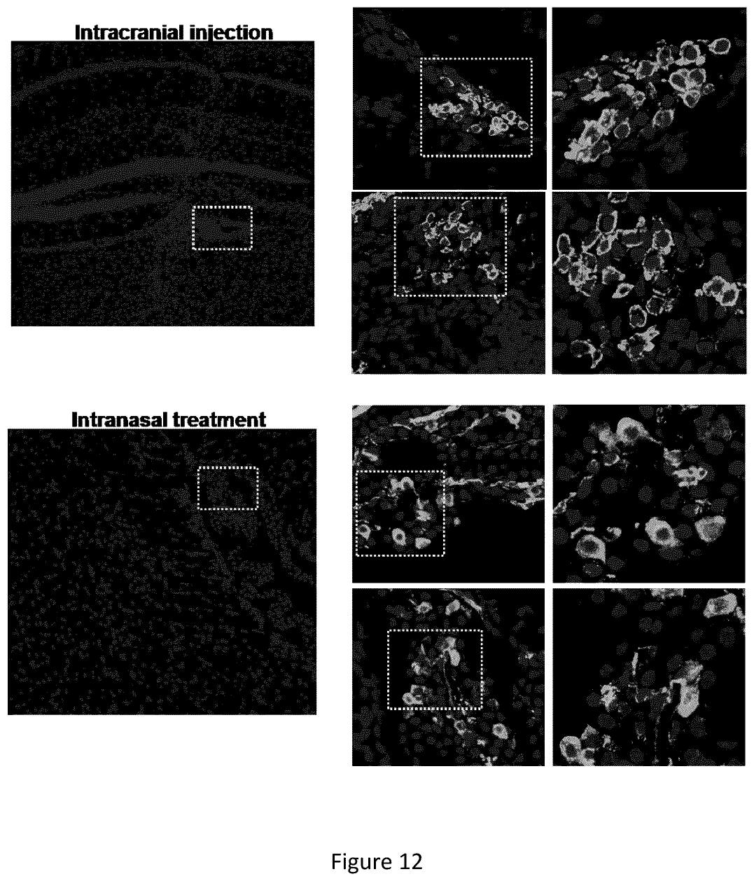

As a result, as illustrated in FIG. 12, it was confirmed that hNTSCs survived well in the brains of the models in which stem cells were transplanted by the IC and IN methods. However, as a result of immunostaining, the survival of hNTSCs was not confirmed in the brain of a model transplanted with cells by the IV method.

### Example 4-3. Confirmation of Ionized Calcium-Binding Adapter Protein-1 (Iba-1) Expression by Intranasal Administration of Inferior Turbinate-Derived Stem Cells (hNTSCs)

The expression of beta-amyloid and a microglial cell marker Iba-1 important for immune responses was confirmed by an immunostaining method 6 weeks after the transplantation of PBS as a control and human inferior turbinate-derived stem cells (hNTSCs) as an experimental group into an Alzheimer's disease mouse animal model overexpressing beta-amyloid by three methods (intracranial injection, nasal dropping, and intravenous injection).

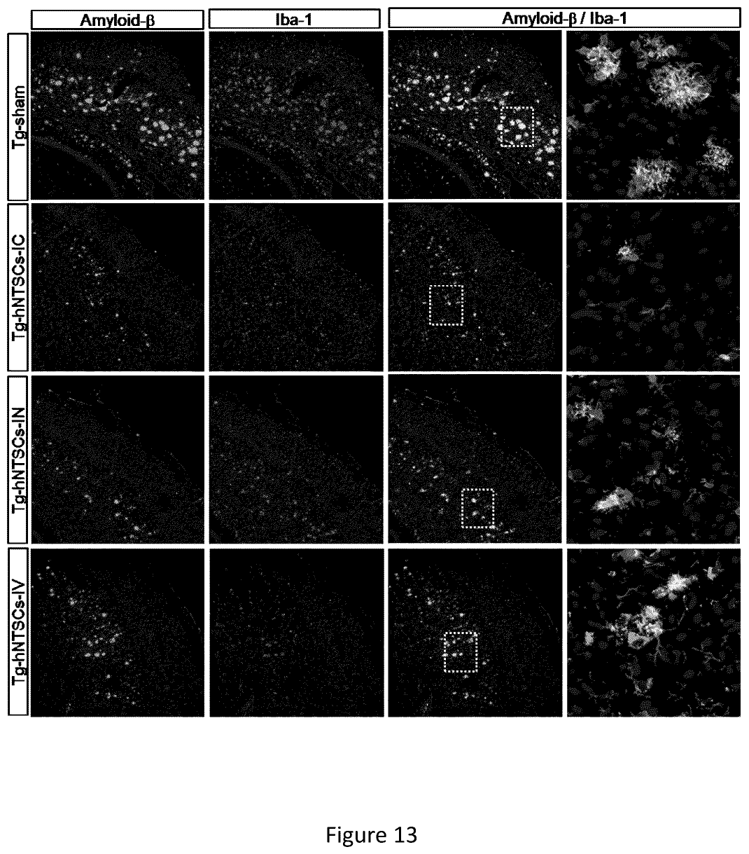

As a result, as illustrated in FIG. 13, it was confirmed that the expression of beta-amyloid and Iba-1 was reduced in the stem cell-transplanted group compared to the PBS-injected group, and the reduction in beta-amyloid expression was more significantly exhibited in the group in which the cells were transplanted by the IC, IN method compared to the IV method.

### Example 4-4. Confirmation of Expression of Neuronal Nuclei (NeuN) by Intranasal Administration of Inferior Turbinate-Derived Stem Cells (hNTSCs)

The expression of beta-amyloid and a nerve cell marker NeuN was confirmed by an immunostaining method 6 weeks after the transplantation of PBS as a control and human inferior turbinate-derived stem cells (hNTSCs) as an experimental group into an Alzheimer's disease mouse animal model overexpressing beta-amyloid by nasal dropping.

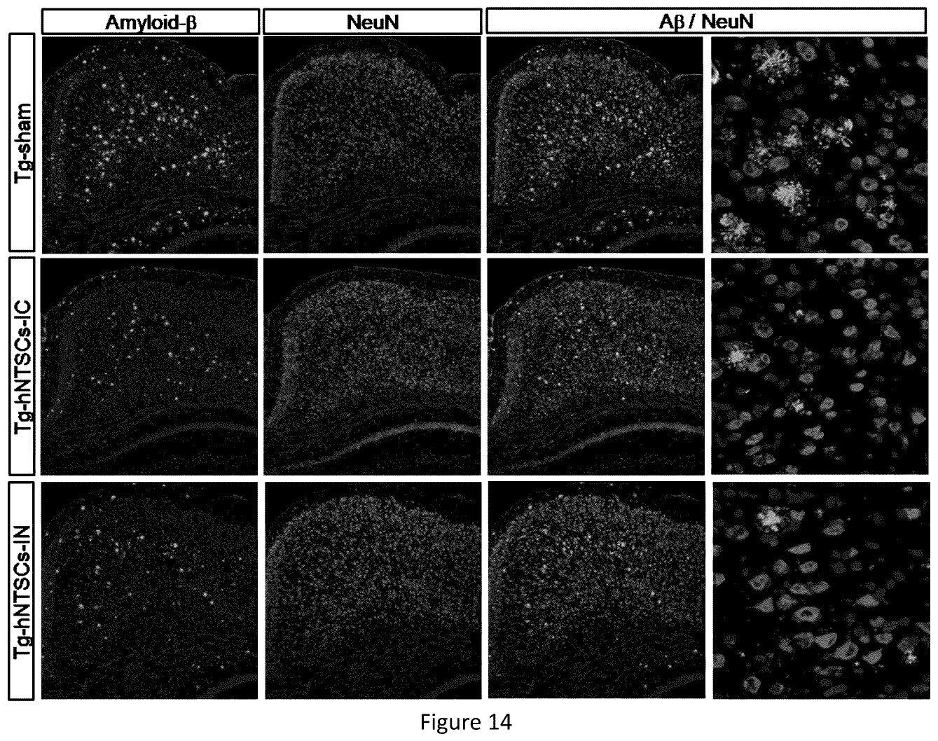

As a result, as illustrated in FIG. 14, it was confirmed that the expression of beta-amyloid was decreased and the expression of nerve cells was significantly increased in the group transplanted with stem cells by the IC and IN methods compared to the PBS-injected group.

Consequently, when stem cells are transplanted by the IC method, the therapeutic effect may be high because cells can be directly delivered to the brain, but it was confirmed that the IN transplantation method is not only safe, but also effective for treatment in consideration of the safety and repeated cell administration of the cell transplantation method.

### Example 5. Exploration of Intramaxillary Administration Route

In addition to the intranasal administration route, routes capable of administering the nasal inferior turbinate-derived mesenchymal stem cells of the present invention were explored.

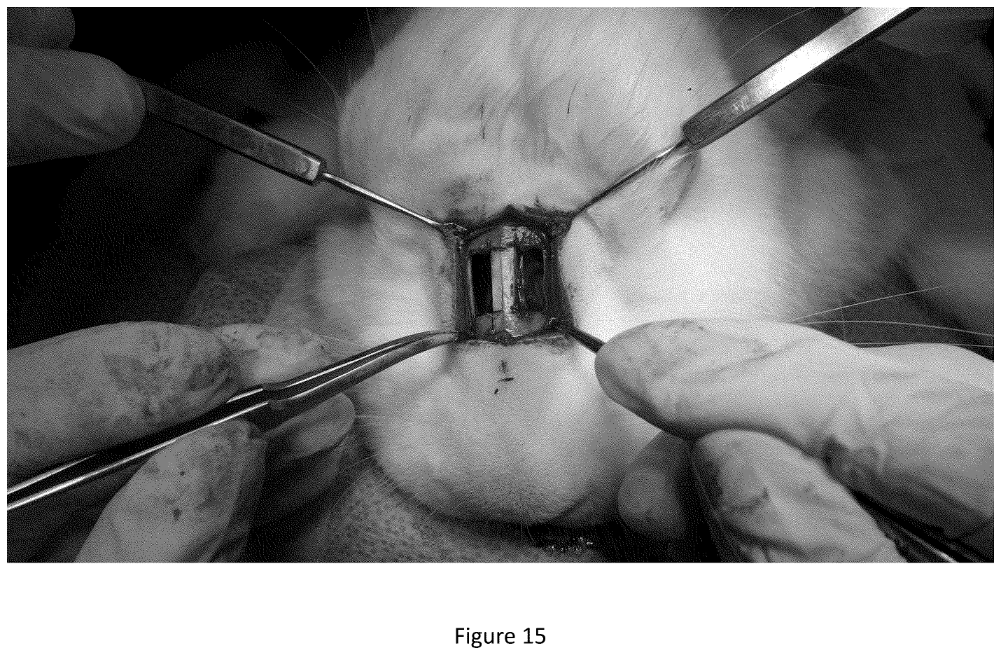

As a result, as illustrated in FIG. 15, the present inventors opened the maxillary bone of a rabbit, and confirmed that a drug can be administered to medium-sized animals such as rabbits through an external transmaxillary approach.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described Examples are only illustrative in all aspects and are not restrictive.

### Industrial Applicability

The present inventors confirmed that when stem cells, and the like are intranasally injected in order to bypass the blood-brain barrier through a peri-olfactory pathway, the stem cells, and the like can be delivered to the brain through the olfactory organ and trigeminal neural pathways, various therapeutic materials including stem cells can be efficiently delivered to the brain and nervous system by the method, and side effects occurring as a result of the entire body being exposed to a drug can be reduced. In addition, intranasal administration to the nasal septum, olfactory mucosal epithelium, superior turbinate adjacent to the olfactory mucosal epithelium, and middle turbinate areas through the nostril or maxillary sinus has an excellent effect of treating brain and nervous system diseases compared to an intravenous administration method of systemic drug exposure, and an intramaxillary injection method that needs to expose the skull during cellular administration, whereas the administration method of the present invention is effective for repeated administration without the need for exposing the skull, and thus has industrial applicability.

1. A method of treating brain and nervous system diseases, the method comprising administering a composition comprising nasal inferior turbinate-derived mesenchymal stem cells as an active ingredient to a subject in need thereof,
wherein the composition is intranasally administered through the nostril or maxillary sinus, and
the nasal cavity comprises one or more areas selected from the group consisting of the nasal septum, olfactory mucosal epithelium, superior turbinate adjacent to the olfactory mucosal epithelium, and middle turbinate areas.

2. The method of claim 1, wherein the composition is characterized by administering nasal inferior turbinate-derived mesenchymal stem cells at 5×10⁵ to 3×10⁷ cells per kg of body weight of a subject to be administered the composition.

3. The method claim 1, wherein the intranasal administration is selected from the group consisting of local administration by injection and administration through a dispenser.

4. The method claim 3, wherein the administration through the dispenser is administration through the form of an aerosol or drop delivery system.

5. The method claim 3, wherein the local administration by injection is administration through subolfactory mucosal injection.

6. The method claim 3, wherein the local administration by injection is administration through intradural injection through the skull base cribriform plate of olfactory mucosa area.

7. The method claim 3, wherein the local administration by injection is administration through intraventricular injection through the skull base cribriform plate and subdural space.

8. The method claim 1, wherein the intranasal administration is partial removal of the maxillary bone of a site to be administered and intranasal administration therethrough.

9. The method claim 1, wherein the brain and nervous system disease is one or more selected from the group consisting of Alzheimer's disease, dementia, Parkinson's disease, stroke, cerebral ischemic stroke, intracerebral hemorrhage, Huntington's disease, multiple sclerosis, spinal cord dysfunction, traumatic brain injury, and encephalitis.

10. The method of claim 1, wherein the composition is a stem cell therapeutic agent.

11-17. (canceled)

18. A method of screening a therapeutic material for brain and nervous system diseases, the method comprising:
(a) administering a candidate material to an animal model subject; and
(b) monitoring the brain tissue of the subject,
wherein the administration is intranasal administration through the nostril or maxillary sinus, and the nasal cavity comprises one or more areas selected from the group consisting of the nasal septum, olfactory mucosal epithelium, superior turbinate adjacent to the olfactory mucosal epithelium, and middle turbinate areas.

19-22. (canceled)

23. The method of claim 18, wherein the intranasal administration is selected from the group consisting of local administration and administration through a dispenser.

24. The method of claim 23, wherein the administration through the dispenser is administration through the form of an aerosol or drop delivery system.

25. The method of claim 23, wherein the local administration by injection is administration through subolfactory mucosal injection.

26. The method of claim 23, wherein the local administration by injection is administration through intradural injection through the skull base cribriform plate of olfactory mucosa area.

27. The method of claim 23, wherein the local administration by injection is administration through intraventricular injection through the skull base cribriform plate and subdural space.

28. The method of claim 18, wherein the intranasal administration is partial removal of the maxillary bone of a site to be administered and intranasal administration therethrough.
